(19) United States
(12) Patent Application Publication
(10) Pub. No.: US 2021/0290190 A1
(43) Pub. Date: Sep. 23, 2021

(54) ANATOMICAL MEASUREMENT DEVICE AND METHOD

(71) Applicant: Rose Medical, inc., Des Plaines, IL (US)

(72) Inventors: Steven Rose, Des Plaines, IL (US); Gregory Rose, Des Plaines, IL (US)

(21) Appl. No.: 16/821,469

(22) Filed: Mar. 17, 2020

(51) Int. Cl. A61B 6/00

(52) U.S. Cl. CPC A61B 6/505

(57) ABSTRACT

An anatomical measurement device and method enables a medical professional to obtain anatomical measurements of a patient. The anatomical measurements may be obtained directly from the patient or from a radiographic image, such as an x-ray, using the measurement device. The measurement device includes a base that includes a linear portion joining branches at each end of the base. The branches may extend perpendicularly to the linear portion. A handle extends at an angle from a first side of the base's linear portion and a protractor arm extends from the opposite side of the base's linear portion. The position and angle of the protractor arm may be adjusted along at least a portion of the length of the base's linear portion. Each of the branches, the linear portion, and the protractor arm may include respective scales to enable the medical professional to take measurements.

### BACKGROUND

As a patient ages, or if a patient is injured, the patient occasionally needs one or more prosthetic implants. For example, a patient may need a partial or total hip replacement. The typical medical procedure for inserting a prosthetic implant requires, in part, that a medical professional determines an appropriate implant size and positioning in relation to the patient's specific anatomy. This is critical to ensure proper fit, as the anatomical dimensions of bones, such as hips, vary from patient to patient. Medical professionals must take multiple pre-operative measurements of the patient, which aid the medical professionals in their decisions for proper preparation of an appropriately sized implant. The pre-operative measurements may involve taking direct measurements of the patient with a measuring device, obtaining measurements from a radiographic image of the patient, such as an x-ray, and some combination of the two. In some instances, these measurements may include length, height, and/or angular measurements. In addition to pre-operative measurements, medical professionals may also take inter-operative measurements (during surgery) and post-operative measurements of the patient and/or the implant, to ensure appropriate size and positioning.

One way to obtain the above-described measurements is to obtain each individual measurement with various scales, templates, protractors, and/or other measuring tools. For example, a medical professional may draw a straight line, using a marker and a straight edge on a pelvic x-ray, at the bottom of the pelvis. Using the line drawn, the medical professional may then measure where the line crosses through the patient's medial cortex of the femoral bones, picking a specific anatomical landmark to reference/gauge the individual leg lengths. Each side/leg is measured separately using a scale.

In another example, a medical professional may measure the abduction angle of an implanted acetabular shell with an acetabular shall template. The medical professional may hold the acetabular shell template, holding the appropriate size shell over the acetabulum on the operative side of the x-ray. For instance, shell templates have all shell sizes, staggered appropriately in numerical order on an 8×11 transparency with all shell sizes on the template displayed with an abduction angle of 45 degrees. When holding the template, the medical professional may reference the line drawn such that, if holding the appropriate sized shell over the acetabulum with the template sheet as a whole approximately straight up and down, the shell implant is at approximately 45 degrees.

An additional example involves measuring a patient's leg offset. For this measurement, the medical professional may use a ruler/scale to measure the distance from the center of the femoral head to the lateral cortex of the proximal femur. Each legs' offset is measured individually with the scale.

### SUMMARY

The present disclosure provides new and innovative devices and methods for obtaining anatomical measurements of a patient. In an example, an anatomical measurement device includes a base, a handle, and a protractor arm. The base extends along a length from a first end to a second end and includes a branch at each of the first end and the second end. The branches extend from the base substantially perpendicular to the length of the base. Each of the branches includes a first scale of measurement indicators. The handle extends from a first side of the base. The protractor arm is adjustably secured to the base and extends from a second side of the base opposite the first side. The protractor arm includes a second scale of measurement indicators. The measurement device is configured such that the protractor arm may (i) translate along at least a portion of the length of the base, (ii) rotate relative to the base, and (iii) remain fixed in position relative to the base.

In an example, a method of obtaining anatomical measurements includes generating a radiographic image of a patient using a radiographic imaging device. An anatomical device is then positioned relative to the radiographic image. The anatomical device includes a base, a handle, and a protractor arm. The base extends along a length from a first end to a second end and includes a branch at each of the first end and the second end. The branches extend from the base substantially perpendicular to the length of the base. Each of the branches includes a first scale of measurement indicators. The handle extends from a first side of the base. The protractor arm is adjustably secured to the base and extends from a second side of the base opposite the first side. The protractor arm includes a second scale of measurement indicators. The measurement device is configured such that the protractor arm may (i) translate along at least a portion of the length of the base, (ii) rotate relative to the base, and (iii) remain fixed in position relative to the base. An anatomical measurement of the patient may be obtained based on the radiographic image relative to the anatomical measurement device.

### BRIEF DESCRIPTION OF THE DRAWINGS

FIG. 1A illustrates an isometric view of a measurement device, according to an aspect of the present disclosure.

FIG. 1B illustrates a front view of the measurement device, according to an aspect of the present disclosure.

FIG. 1C illustrates a side view of the measurement device, according to an aspect of the present disclosure.

FIG. 1D illustrates a bottom view of the measurement device, according to an aspect of the present disclosure.

FIG. 1E illustrates a magnified view of the respective scales of the measurement device shown in FIG. 1B, according to an aspect of the present disclosure.

FIG. 2 illustrates a measurement device positioned against a radiographic image, according to an aspect of the present disclosure.

### DETAILED DESCRIPTION

The present disclosure relates to new and innovative devices and related methods for obtaining anatomical measurements of a patient. In a preferred embodiment, the measurement device includes a handle extending at an angle away from a generally U-shaped base portion. For example, the U-shaped base portion may include both a linear portion and a branch at each of the linear portion's terminal ends. In various embodiments, the branches may be angled relative to the linear portion. In one embodiment, each of the branches are disposed at 90 degree angles to the linear portion.

Each of the branches includes a scale that may be used to measure a length and/or height of an anatomical feature of the patient. The base's linear portion may, likewise, include a scale for taking various measurements of anatomical features. The measurement device further includes a protractor arm that is adjustably secured to the base portion. The protractor arm is configured to slide along at least a segment of the linear portion of the base, and may also rotate relative to the base portion. Rotating the protractor arm enables a medical professional to take an angular measurement of various anatomical features of the patient. Specifically, for example, the protractor arm includes a scale, and the position of the base's linear portion in relation to the scale determines the angular measurement reading. Advantageously, the protractor arm may be fixed in place, relative to the base, so that it does not move as the device is moved.

The provided measurement device accordingly enables the medical professional to take a variety of anatomical measurements of the patient in a simple and efficient manner with a single tool. For example, the medical professional may hold the measurement device's handle and position the measurement device relative to the patient in multiple positions and/or rotate the protractor arm to take various anatomical measurements. In another example, the medical professional may hold the measurement device's handle and position the base flat against a radiographic image of the patient. In an embodiment, the measurement device is transparent or semi-transparent so that the medical professional may take anatomical measurements using the scales on the device in relation to the radiographic image without obstructing the view of the radiographic image with the measurement device. In either of the examples described above, the medical professional may adjust the protractor arm by sliding it, rotating it, or both, or may fix the protractor arm in place. The measurement device enables the medical professional to hold the measurement device against the radiographic image with one hand, and simultaneously take notes or point out anatomical features with the other hand, thus increasing the overall ease and efficiency of taking the measurements as compared to obtaining individual measurements with various scales, templates, protractors, and/or other measuring tools.

In certain instances, the presently disclosed measurement device may be particularly adapted for measuring the anatomy of the patient's hips. For example, the scales on the measurement device's branches may enable the medical professional to determine the patient's leg length. Similarly, the one or more scales on the measurement device's linear portion may enable the medical professional to determine the patient's leg offset. Therefore, the medical professional may quickly and efficiently take leg length and leg offset measurements with minimal to no repositioning of the measurement device. Additionally, the measurement device may be sized with a width that is greater than a width between the medial cortex of each of the patient's respective femurs. In such an example, when holding the measurement device against a radiographic image, the device extends to both sides of the patient's hips. Thus, the medical professional may take measurements at both sides of the patient's hips with minimal to no repositioning of the measurement device. The medical professional may also use the measurement device to visually compare measurements between the sides, such as right leg offset vs. left leg offset. For instance, the medical professional is able to compare measurements of the patient's operative or implant side with measurements of the patient's non-operative side.

The measurement device also enables the medical professional to easily measure an abduction angle of an acetabular shell implant. For instance, the measurement device's width and adjustable protractor arm enables the medical professional to position the measurement device against a radiographic image such that the base's linear portion is perpendicular to the patient's spine or ischial tuberosity, while adjusting the protractor arm to measure the abduction angle. Positioning the measurement device perpendicular to the spine or ischial tuberosity, when taking the angular measurement, helps ensure an accurate and consistent angular measurement reading. Additional advantages of the presently disclosed devices and methods will be apparent to one having skill in the art, in light of the following description of the figures.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

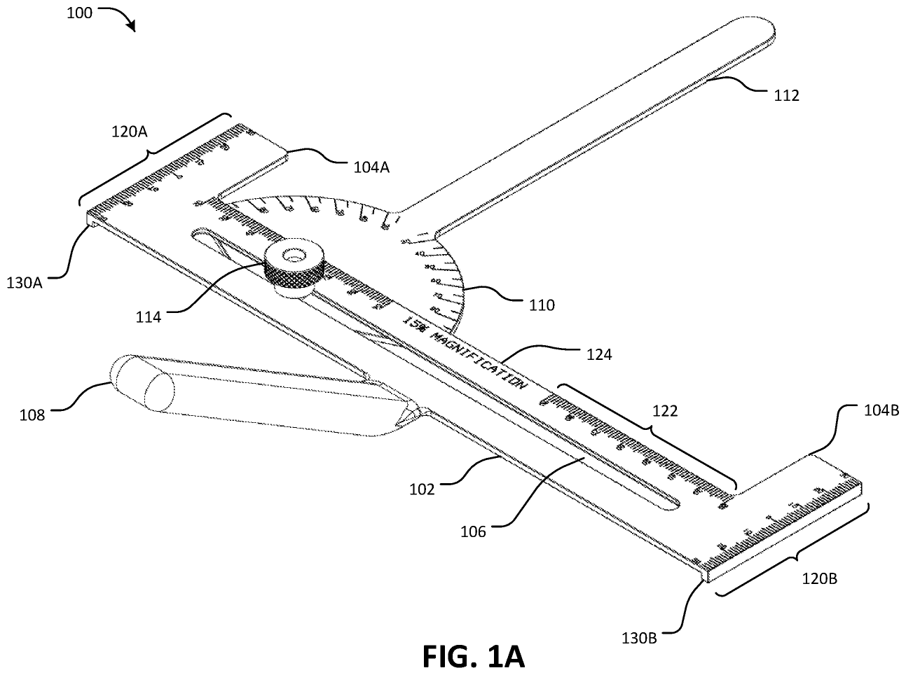

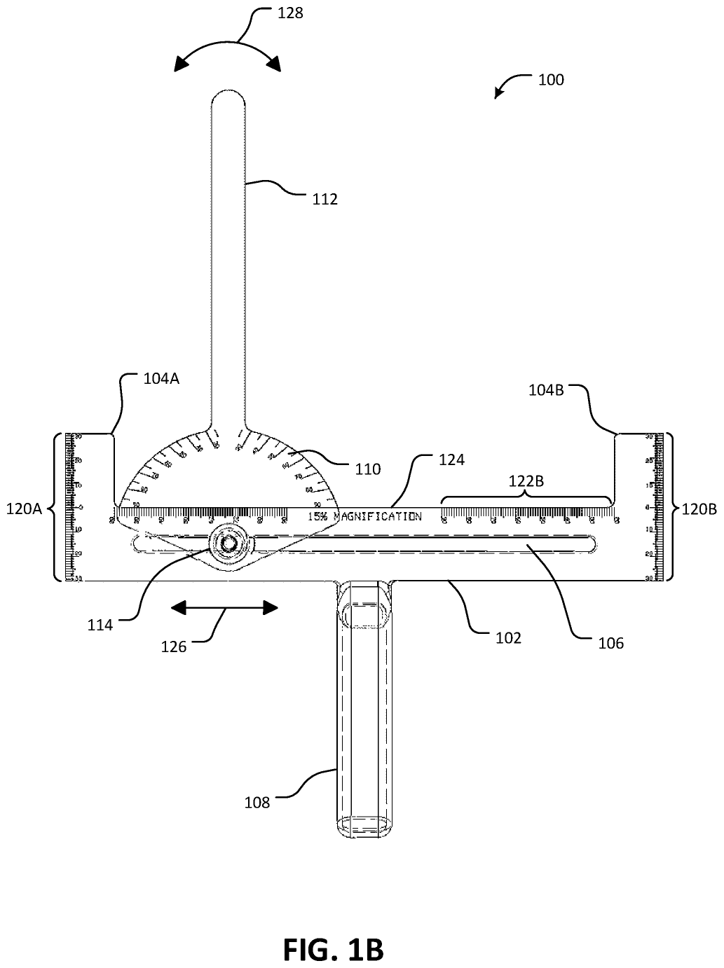

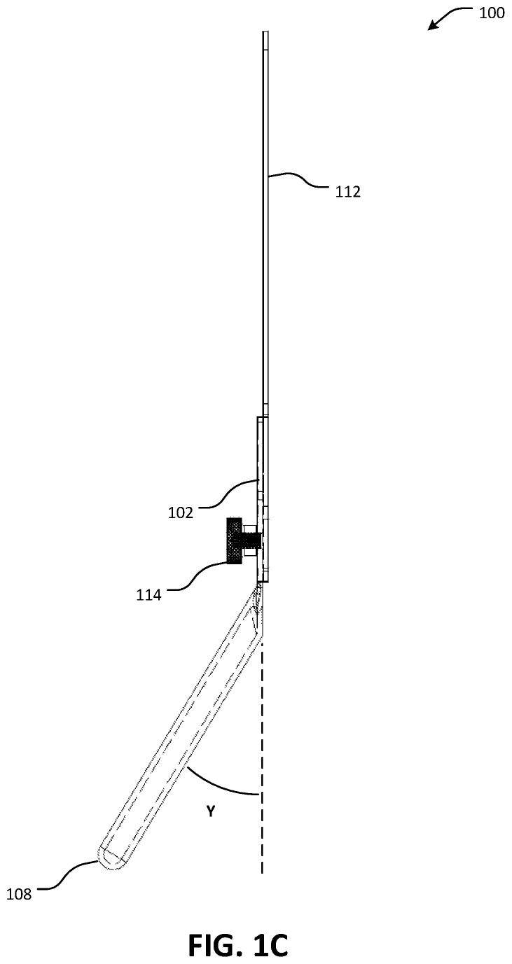

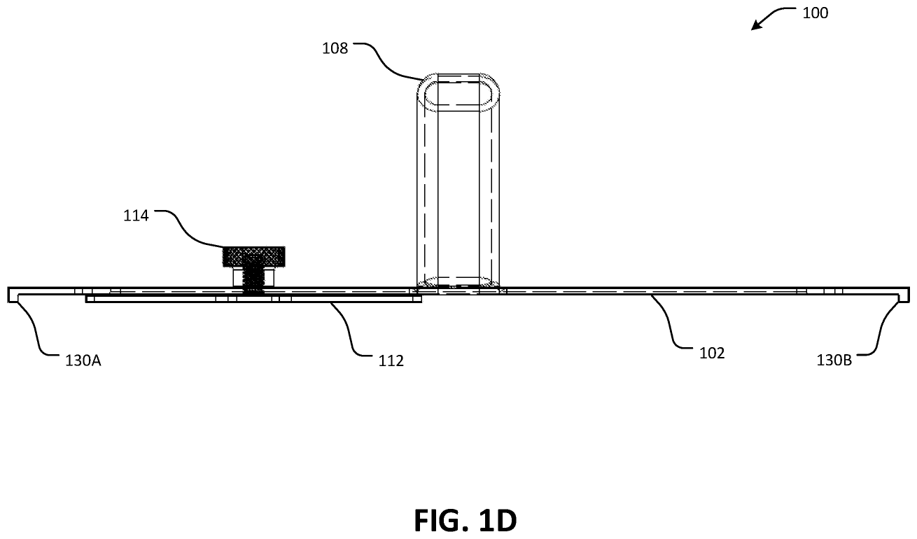

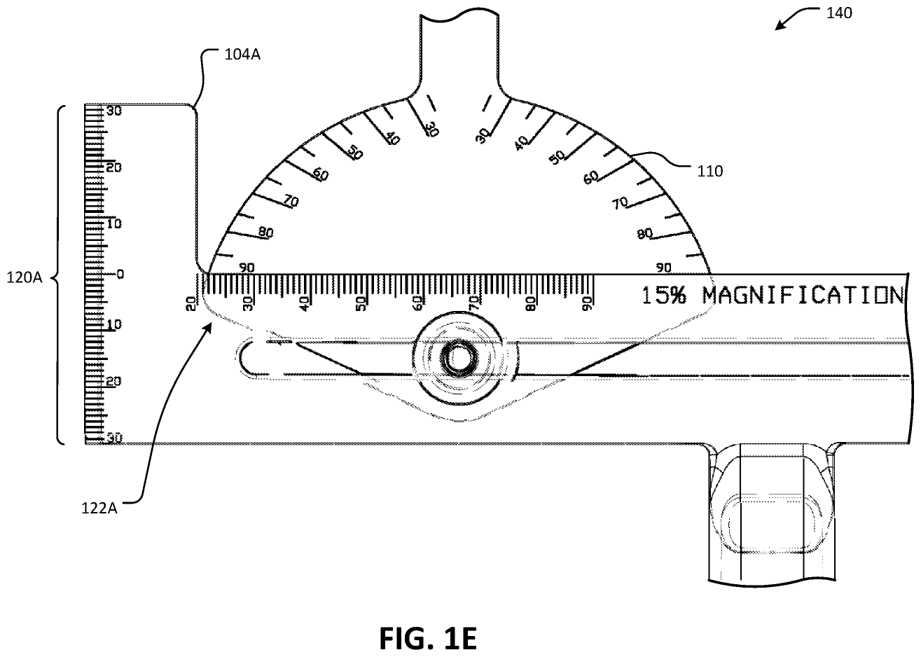

FIGS. 1A to 1E illustrate multiple different views of an exemplary measurement device 100, according to an aspect of the present disclosure. FIG. 1A illustrates a perspective view of the measurement device 100. The measurement device 100 includes a base 102 that extends along a length from a first end to a second end. In some embodiments, the base 102 includes an opening 106 that extends along at least a portion of the length of the base 102. For example, opening 106 may be perpendicular with base 102. The base 102 includes two branches 104A, 104B one of which is disposed at each end of base 102 and a linear portion that joins the branches 104A, 104B. In an embodiment, each branch 104A, 104B is formed integrally with the linear portion and the base 102, such that base 102, branch 104A, and branch 104B are a single component. In other embodiments, each branch 104A, 104B may be a separate component coupled to the linear portion of the base 102. Each branch 104A, 104B may extend from the base 102 at an angle substantially equal to 90 degrees with respect to the linear portion of the base 102. The base 102 may further include protrusions 130A, 130B, disposed along a height of each respective branch 104A, 104B, which will be discussed in more detail with respect to FIG. 1D.

Each branch 104A, 104B may include a scale 120A, 120B of measurement indicators. For example, each measurement indicator may indicate millimeters or inches. Similarly, the base 102 may include one or more scales 122A and 122B of measurement indicators on its linear portion as illustrated by FIG. 1E. The edge of the linear portion of the base 102 that includes the scales 122A, 122B may be referred to as the measuring edge 124 of the linear portion.

The measurement device 100 additionally includes a handle 108 extending from the base 102. Specifically, the handle 108 extends at an angle from the base 102, which is discussed in more detail in connection with FIG. 1C. In some aspects of the present disclosure, such as the one illustrated, the handle 108 extends from a side of the base 102 that is opposite of the side of the base 102 from which the branches 104A, 104B extend. In other aspects, the handle 108 and the branches 104A, 104B may extend from the same side of the base 102.

The measurement device 100 may also include a protractor arm 112. The protractor arm 112 includes a scale 110 of measurement indicators. For example, each measurement indicator may indicate a quantity of angular degrees. The scale 110 will be discussed in more detail in connection with FIG. 1E. The protractor arm 112 may be adjustably secured to the base 102. For example, the protractor arm 112 may include a male threaded member and may be secured to the base 102 with a female threaded knob 114, a flat washer and a curved disc spring. The male threaded portion of the protractor arm 112 may be placed through the opening 106 of the base 102 and the female threaded portion of the knob 114 may screw onto the male threaded portion. When the knob 114 is fully tightened, the curved disc spring is flattened, placing full compressive force on the base 102 between the knob 114 and the protractor arm 112. In this configuration, the protractor arm 112 is fixed in place. When the knob 114 is loosened, the curved disc spring bends, and less compressive force is placed on the base 102 between the knob 114 and the protractor arm 112. The more the knob 114 is loosened, the more freely the protractor arm 112 may be adjusted relative to the base 102. In other examples, the protractor arm 112 may include the female threaded end and the knob 114 may include the male threaded end.

FIG. 1B illustrates a front view of the measurement device 100 and, in particular, shows that the protractor arm 112 may be adjusted relative to the base 102. As indicated by the double-sided straight arrow 126, the protractor arm 112 may translate along at least a portion of the length of the base 102. For instance, the protractor arm 112 translates along the portion of the base 102 that includes the opening 106. In at least one example, the opening 106 has a length equal to 9.5 inches. As indicated by the double-sided curved arrow 128, the protractor arm 112 may also rotate relative to the base 102. For instance, the protractor arm 112 rotates relative to the base 102, by pivoting about knob 114. This rotation enables the medical professional to measure an angle between the protractor arm 112 and the base 102.

FIG. 1C illustrates a side view of the measurement device 100, and in particular shows an angle Y at which the handle 108 extends from the base 102. The handle 108 extends from the base 102 at a sufficient angle Y to enable the medical professional to hold the handle while the protrusions 130A and 130B are positioned flat against a surface, such as a radiographic image. For example, the angle Y may be between 20 to 135 degrees. Preferably, the angle Y is between 20 to 70 degrees. More preferably, the angle Y is between 30 to 45 degrees. Most preferably, the angle Y is 30 degrees. In aspects in which the angle Y is less than 90 degrees, the medical professional typically holds the handle 108 with an underhand grip. In aspects in which the angle Y is equal to or greater than 90 degrees, the medical professional typically holds the handle 108 with an overhand grip. An underhand grip may be preferable in various instances so that the medical professional's hand and arm are out of view of the respective scales when the medical professional is taking anatomical measurements. Additionally, in at least one example, the handle 108 is approximately five inches in length. In other instances, the handle 108 may be other suitable lengths that are sufficient for the medical professional to hold and position the measurement device 100.

FIG. 1D illustrates a bottom view of the measurement device 100 and, in particular, shows the protrusions 130A, 130B in relation to the other components of the device 100. The protrusions 130A, 130B extend from the base 102, such that the protrusions 130A, 130B may contact a surface that the measurement device 100 is positioned against. For example, when the measurement device 100 is positioned against a radiographic image, the protrusions 130A, 130B may contact the radiographic image and may prevent the rest of the base 102 and the protractor arm 112 from contacting the radiographic image. By preventing the protractor arm 112 from contacting the radiographic image, the protrusions 130A, 130B enable the protractor arm 112 to easily translate and/or rotate when the measurement device is positioned against a radiographic image. For instance, the protrusions 130A, 130B reduce or avoid friction between the protractor arm 112 and the radiographic image. The protrusions 130A, 130B may also advantageously prevent damage to the radiographic image that could be caused by rubbing the protractor arm 112 against the radiographic image as the protractor arm 112 is translated and/or rotated.

FIG. 1E illustrates a magnified view 140 of the respective scales of the measurement device 100 shown in FIG. 1B, according to an aspect of the present disclosure. It should be appreciated that only the scale 120A and the scale 122A are illustrated due to the magnified nature of the figure, and that the following respective descriptions apply equally to the respective scales 120A and 120B and to the respective scales 122A and 122B. In some instances, the measurement indications are proportionally scaled on the respective scales. For example, the measurement indications may be scaled at 15% magnification. X-ray images are typically generated at 15% magnification and thus, in such examples, the scaled measurement indications enable the medical professional to take measurements on an x-ray image without the need for any magnification-based conversions. In other examples, the measurement indications may be scaled at other suitable magnification levels, or may not be scaled.

In some aspects, such as the one illustrated, the respective scales 120A, 120B may be positioned along the outermost edges of each branch 104A, 104B. In other instances, the respective scales 120A, 120B may extend along the respective widths of the branches 104A, 104B, or may be positioned on the opposite side of each branch 104A, 104B (e.g., positioned on the side closer to the center of the device 100). The measurement indications of the scales 120A, 120B may, in various instances, include a centered zero position and increasing indications on either side. For example, as illustrated in FIG. 1E, the measurement indications of the scale 120A include thirty indications, each representative of a millimeter, on either side of a zero position. In other examples, the scales 120A, 120B may include more or less than sixty measurement indications. The branches 104A, 104B may include added height to accommodate additional measurement indications.

The centered zero position of the scales 120A, 120B enables a medical professional to measure lengths in either direction of the centered zero position with respect to an anatomical landmark that the medical professional chooses to reference. For example, when measuring a patient's leg lengths with the measurement device 100 on an x-ray, positioning the measurement device 100 with the top lateral edge at the bottom of the pelvis and perpendicular to the spine results in the measurement device 100 crossing through the medial cortex. The medical professional may then reference where the measurement device 100 crosses through the lesser trochanter. For example, due to many variables that can affect the clarity and image of an x-ray, the bottom/distal end of the lesser trochanter is more visible and defined in a patient's pelvic x-ray, making it a better anatomical landmark to reference for measuring the patient's leg lengths. In this example, being able to measure going down from the centered zero position is less difficult and thus enables the medical professional to read measurements faster than having to subtract measurement numbers on a scale without a centered zero position.

In some aspects of the present disclosure, the base 102 may include a single scale that extends along the entire length of the base 102. In other aspects, the base 102 may include more than one separate scale, such as the scale 122A and the scale 122B. As shown in FIG. 1E, the measurement indications of the scale 122A may, in various aspects, measure from the outermost edge of the branch 104A. For example, the lowest measurement indication on the scale 122A is twenty millimeters at the innermost edge of the branch 104A, and the width of the branch 104A is twenty millimeters. Refraining from including the first twenty millimeter indications on the scale 122A may help maintain clarity between the scale 120A and the scale 122A.

The angular scale 110 includes measurement indications that indicate an angle between the base 102 and the protractor arm 112. In various aspects, such as the one illustrated, the scale 110 may include two symmetric sets of measurement indications such that an angle may be measured irrespective of whether the protractor arm 112 is rotated to the right or to the left. The scale 110 may enable the medical professional to measure angles between 25 to 90 degrees in various instances. In some examples, the scale 110 may include additional measurement indications than that illustrated in FIG. 1E, so that the angle may be measured more precisely.

In various aspects of the present disclosure, the measurement device 100 is constructed of a plastic material or other polymer. In some examples, the measurement device 100 is constructed using additive manufacturing. The measurement device 100 may be transparent or semi-transparent, such that the medical professional can see through the measurement device 100 when held up against a radiographic image and can take measurements of anatomic features that would otherwise be covered by the measurement device 100.

In certain examples, the measurement device 100 may be particularly adapted to measure the anatomical features of the patient's hips. For instance, the measurement device 100 may have a width between the outermost edges of the branches 104A, 104B that is equal to or greater than a width between the respective medial cortex of each respective femur of the patient. In at least one example, the measurement device 100 has a width equal to approximately eleven inches.

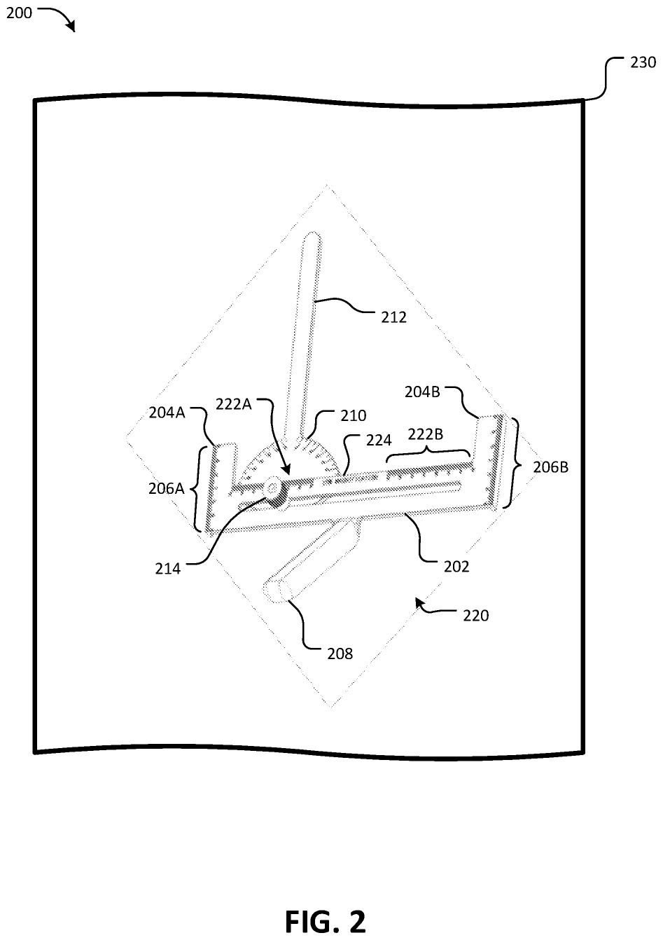

The medical professional may utilize the measurement device 100 to take a variety of anatomical measurements of the patient. For instance, as described above, the medical professional generates a radiographic image, such as an x-ray, of the patient (e.g., the patient's pelvic region) using a radiographic image device. The medical professional positions the measurement device 100 relative to the radiographic image in order to obtain measurements. FIG. 2 shows an example method 200 of positioning a measurement device 220 against a radiographic image 230. The measurement device 220 may be configured according to the description provided above with regard to the measurement device 100. The measurement device 220 is positioned such that the base 202, including the protrusions extending from the base 202, is flat against the radiographic image 230. The medical professional then obtains anatomical measurements based on the anatomical features in the radiographic image 230 relative to the measurement indications on the respective scales of the measurement device 220. In some instances, the medical professional takes additional anatomical measurements of the patient by measuring them directly on the patient, prior to generating the radiographic image.

In one example method, the medical professional obtains anatomical measurements of the patient's hips from the generated radiographic image 230. The medical professional positions the measurement device 220 relative to the radiographic image 230 such that the measuring edge 224 of the linear portion of the base 202 is below the patient's pelvis at a distal edge of the patient's ischium and such that the measuring edge 224 is perpendicular to the patient's spine or ischial tuberosity. In this example method, the measurement device 220 is particularly adapted for obtaining anatomical measurements of the patient's hips, as described above, and thus the measurement device 220 overlaps the medial cortex of both of the patient's femurs on the radiographic image 230. In this position, the medical professional may obtain various anatomical measurements. In some instances, obtaining the measurements may require adjusting the positioning of the measurement device 220 while maintaining the above-described positioning.

For example, the medical professional may obtain a leg length measurement. The medical professional may, for instance, obtain the leg length measurement by measuring a distance between (1) a point where the upper edge (e.g., the edge with the scale) of the linear portion of the base 202 crosses the patient's medial cortex of the femur, and (2) the top/proximal cortex of the greater trochanter of the patient's femur. The medical professional uses the scales 206A, 206B on the respective branches 204A, 204B to measure this distance on each respective side of the patient's hips. The medical professional may quickly compare the leg length measurements of each respective side since the measurement device 220 extends to both sides and enables the medical professional to take measurements of both sides at the same time, or at least quickly one after the other. The leg length measurement may be taken pre-operatively.

In comparison, a medical professional using typical measuring tools for leg length measurements would use a straightedge to draw a line, parallel to the pelvis and perpendicular to the spine, from the top edge of the greater trochanter across towards the greater trochanter of the other leg. The medical professional would then measure the distance between the line drawn and the greater trochanter depending on where the line crosses through the cortex of the proximal femur on the opposite leg. Measuring the distance from the line drawn and up to the top of the greater trochanter would give the approximate length of how much shorter the opposite leg is. Alternatively, the line drawn across towards the opposite leg's greater trochanter may not cross through the cortex of the opposite leg's proximal femur. In such instances, the medical professional measures the distance from the line drawn and down to the top of the greater trochanter to obtain an approximate length for how much longer the opposite leg is.

In another example, the medical professional may obtain a leg offset measurement with the measurement device 220. The medical professional obtains the leg offset measurement by measuring a distance between (1) a center of a femoral head, and (2) a lateral edge of a femur. The medical professional uses the scales 222A, 222B on the linear portion of the base 202 to measure this distance on each respective side of the patient's hips. The medical professional may quickly compare the leg offset measurements of each respective side since the measurement device 220 extends to both sides and enables the medical professional to take measurements of both sides at the same time, or at least quickly one after the other. The leg offset measurement may be taken pre-operatively.

The medical professional may also take measurements inter-operatively when performing a procedure, such as a hip replacement procedure. For example, the medical professional obtains a femoral broach depth measurement from an x-ray generated inter-operatively, by measuring a distance between (1) the proximal tip of the patient's greater trochanter and (2) the top/proximal shoulder of the patient's femoral broach. The medical profession uses one or more of the scales 206A, 206B on the branches 204A, 204B to measure this distance. In certain instances, the medical professional only obtains a femoral broach depth measurement on one of the patient's legs. For example, a patient's opposite, non-operative, leg may have never undergone reconstruction and there is not a femoral stem implant on the opposite leg with which to compare measurements. In another example, a patient may have had the opposite leg previously reconstructed, but with an implant that is manufactured by a different company and manufacturer than the implant for the leg that the medical professional is reconstructing. Implants from different manufacturers may vary in their designs, which may affect the depth at which the broach/femoral stem is seated, thus rendering measurement incomparable between the implants.

In other instances, the patient's opposite leg may have been previously reconstructed with the same implant, manufactured by the same company and manufacturer. In such instances, the medical professional may obtain femoral broach depth measurements on each of the patient's legs. The opposite leg may be a good reference for the medical professional to know roughly the broach depth that could be obtained, which can aid the medical professional in obtaining matching leg lengths when carrying out the surgical procedure.

In another example, the medical professional may obtain an abduction angle measurement of an acetabular shell implant from an x-ray generated inter-operatively. To obtain the abduction angle measurement the medical professional uses the protractor arm 212. For instance, the medical professional may position the measurement device 220 such that the measuring edge 224 is perpendicular to the patient's spine or ischial tuberosity and may adjust the lateral positioning and/or angle of the protractor arm 212. The protractor arm 212 is aligned to be parallel with the face/hemisphere of the acetabular shell. When the protractor arm 212 is approximately parallel to the acetabular shell, the angle indicated on the scale 210 is the abduction angle that the implanted acetabular shell is seated at. The configuration of the measurement device 220 enables the medical professional to hold the handle 208 with one hand to position the measurement device 220 and adjust the protractor arm 212 with the medical professional's second hand at the same time.

The medical professional may also take each of the above-described measurements post-operatively after performing a procedure, such as a hip replacement procedure. The medical professional may obtain a measurement from an x-ray generated after performing the procedure. For example, the medical professional may measure a femoral stem implant's depth to ensure that the implant is properly seated in the patient's femoral canal. The medical professional may use one or more of the scales 206A, 206B on the branches 204A, 204B to obtain this measurement.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated.

The invention is claimed as follows:

1. An anatomical measurement device comprising:
a base extending along a length from a first end to a second end, the base including a branch at each of the first end and the second end, wherein the branches extend from the base substantially perpendicular to the length of the base, and wherein each branch includes a first scale of measurement indicators;
a handle extending from a first side of the base; and
a protractor arm adjustably secured to the base, the protractor arm extending from a second side of the base opposite the first side, wherein the protractor arm includes a second scale of measurement indicators, and
wherein the device is configured such that the protractor arm may (i) translate along at least a portion of the length of the base, (ii) rotate relative to the base, and (iii) remain fixed in position relative to the base.

2. The anatomical measurement device of claim 1, wherein the base includes an opening extending along at least a portion of the length of the base.

3. The anatomical measurement device of claim 2, wherein the protractor arm is adjustably secured to the base with a knob.

4. The anatomical measurement device of claim 1, wherein the branches extend from the second side of the base.

5. The anatomical measurement device of claim 1, wherein the second side of the base includes a third scale of measurement indicators.

6. The anatomical measurement device of claim 1, wherein the measurement indicators of the first scale are scaled at 15% magnification.

7. The anatomical measurement device of claim 6, wherein the measurement indicators of the first scale include a total measurement indication of at least 60 millimeters.

8. The anatomical measurement device of claim 1, wherein the handle extends from the base at an angle between 20 to 135 degrees relative to a plane that includes the first and second sides of the base.

9. The anatomical measurement device of claim 1, wherein the device is constructed of a plastic material.

10. The anatomical measurement device of claim 1, wherein the device is transparent.

11. The anatomical measurement device of claim 1, wherein each of the branches includes a respective protrusion along a height of the respective branch.

12. The anatomical measurement device of claim 1, wherein the device is sized having a width such that the respective ends of the device extend to at least the medial cortex of each respective femur of a patient.

13. A method of obtaining anatomical measurements of a patient comprising:
generating a radiographic image of a patient using a radiographic imaging device;
positioning an anatomical measurement device relative to the radiographic image, wherein the anatomical measurement device includes:
a base extending along a length from a first end to a second end, the base including a branch at each of the first end and the second end, wherein the branches extend from the base substantially perpendicular to the length of the base, and wherein each branch includes a first scale of measurement indicators,
a handle extending from a first side of the base, and
a protractor arm adjustably secured to the base, the protractor arm extending from a second side of the base opposite the first side, wherein the protractor arm includes a second scale of measurement indicators, and
wherein the device is configured such that the protractor arm may (i) translate along at least a portion of the length of the base, (ii) rotate relative to the base, and (iii) remain fixed in position relative to the base; and
obtaining an anatomical measurement of the patient based on the radiographic image relative to the anatomical measurement device.

14. The method of claim 13, wherein the radiographic image includes a radiographic image of the hips of the patient, and wherein positioning the anatomical measurement device relative to the radiographic image includes positioning the anatomical measurement device such that (i) each branch is below the pelvis at a distal edge of the ischium and (ii) each branch is perpendicular to the ischial tuberosity.

15. The method of claim 13, wherein the radiographic image includes a radiographic image of the hips of the patient, and wherein the anatomical measurement device is sized having a width such that, when positioned relative to the radiographic image, the anatomical measurement device overlaps the medial cortex of both femurs of the patient.

16. The method of claim 13, wherein the radiographic image includes a radiographic image of at least one hip of the patient, and wherein obtaining an anatomical measurement includes obtaining a length measurement from the center of a femoral head to a lateral edge of a femur.

17. The method of claim 13, wherein the radiographic image includes a radiographic image of at least one hip of the patient, and wherein obtaining an anatomical measurement includes obtaining a length measurement from a proximal tip of a greater trochanter to a proximal shoulder of a femoral broach.

18. The method of claim 13, wherein the radiographic image includes a radiographic image of at least one hip of the patient, and wherein obtaining an anatomical measurement includes adjusting the protractor arm to measure an angle of an acetabular shell.

19. The method of claim 18, wherein measuring the angle of the acetabular shell includes positioning the base of the anatomical measurement device such that the base is perpendicular to the spine or ischial tuberosity of the patient.

20. The method of claim 13, further comprising, prior to generating the radiographic image, obtaining anatomical measurements of a patient based on the patient relative to the anatomical measurement device.
